(19) United States
(12) Patent Application Publication
(10) Pub. No.: US 2024/0366632 A1
(43) Pub. Date: Nov. 7, 2024

(54) METHODS FOR THE TREATMENT OF OCULAR ROSACEA

(71) Applicants: INSTITUT NATIONAL DE LA SANTÉ ET DE LA RECHERCHE MÉDICALE, Paris (FR); ASSISTANCE PUBLIQUE-HÔPITAUX DE PARIS (APHP), Paris (FR); SORBONNE UNIVERSITÉ, Paris (FR); UNIVERSITÉ PARIS CITÉ, Paris (FR)

(72) Inventors: Francine BEHAR-COHEN, Paris (FR); Min ZHAO, Paris Cedex 06 (FR)

(21) Appl. No.: 18/684,010

(22) PCT Filed: Aug. 31, 2022
(86) PCT No.: PCT/EP2022/074208

§ 371 Date: Feb. 15, 2024

(30) Foreign Application Priority Data
Aug. 31, 2021 (EP) 21306177.3

(51) Int. Cl. A61K 31/585; A61K 31/4375; A61K 31/573; A61K 31/575; A61P 27/02; C12N 15/113

(52) U.S. Cl. CPC A61K 31/585; A61K 31/4375; A61K 31/573; A61K 31/575; A61P 27/02; C12N 15/1138; C12N 2310/11; C12N 2310/14

(57) ABSTRACT

Ocular Rosacea (OR) is a chronic inflammatory and neurovascular diseases of the ocular surface and eyelids. associated with abnormal tear film lipids that can lead to corneal neovascularization, loss of transparency and ulceration. Here, the inventors show that the combination of mineralocorticoid receptor blockade in association with local ocular glucocorticoids that have high GR binding affinity, have superior effects as compared to MR blockade alone, without the side effects of glucocorticoids on corneal wound healing. The combination of MR antagonist and low dose of a GR activator further reduces corneal edema, corneal neovascularization and improves corneal wound healing. The combination of MR antagonist and triamcinolone that has a strong GR binding affinity reinforces the beneficial effects of MR antagonists. Finally, the inventors show that MR is overexpressed in ocular surface tissues and Meibomian glands of patients with ocular rosacea, and that transgenic rats that over express the human MR have molecular markers in their meibomian glands, similar to those of patients with OR. Thus, MR antagonists and GR agonist with strong GR affinity is a suitable combination for the treatment of OR.

### FIELD OF THE INVENTION

The present invention is in the field of medicine, in particular ophthalmology.

### BACKGROUND OF THE INVENTION

Rosacea, a common facial chronic inflammatory skin disease characterized by redness, telangiectasia, papules and pustules affects approximately 10% of the population, and 10 to 50% of patients have ocular surface involvement (1,2). Ocular Rosacea (OR) is a chronic inflammatory and neurovascular diseases of the ocular surface and eyelids, associated with abnormal tear film lipids that can lead to corneal neovascularization, loss of transparency and ulceration. OR, even in its mild presentations, affect patient's quality of life, can threaten vision and is underdiagnosed and so far, incurable.

The ocular surface comprises the tear film, the cornea and conjunctiva and the inner faces of the eyelids that function in a coordinated manner to maintain homeostasis, corneal lubrication and transparency despite direct exposure to the environmental aggressions. OR is a multifactorial disease of the ocular surface, initiated or aggravated by endogenous and exogenous triggering factors, including ultraviolet (UV) irradiation, infections, and psychosocial stress. It results from innate and adaptive immune dysfunction, neurovascular dysregulation (3-5) and neurogenic inflammation (4,6). In the eyelids, Meibomian glands (MG) that produce the lipid layer of the tear film are under autonomous nerve regulation. Meibomian gland dysfunction (MGD), characterized by an alteration of the tear lipid composition is associated with OR (7). The triggering factors may cause ocular symptoms and damages by over-activation of the autonomous nerves and/or by the release of neurovascular and neuroimmune active neuropeptides (ex. substance P, neuropeptide Y, calcitonin gene-related peptide (CGRP), VIP, NO) (8,9), which contribute to the neurogenic inflammation. Accumulating evidence also points to activation of the toll like receptor (TLR) 2 (10) and transient receptor potential (TRP) ion channels (11,12) with subsequent release of inflammatory mediators from multiple cell types including keratinocytes, mast cells, neurons, endothelial cells, macrophages, fibroblasts and Th1/Th17 cells (8,13,14) as a primary or secondary causative mechanism of skin rosacea.

Both skin and eye tissues express enzymes that contribute to local homeostasis of corticosteroids (15,16), which have been incriminated in the pathogenesis of rosacea. Indeed, UVB exposure and psychological stress stimulates excessive corticosteroidogenesis (15) and interestingly chronic treatment with local glucocorticoids (GCs), despite their anti-inflammatory effects, can induce rosacea-like dermatitis (17). However, the mechanisms of corticoids pathogenic effects remain poorly understood. GCs bind to the glucocorticoid receptor (GR) and also to the mineralocorticoid receptor (MR) with high affinity. Both GR and MR are expressed in the skin, cornea, conjunctiva and the MG as shown below. Of note, the activity of the MR-protecting enzyme 11-β-hydroxysteroid dehydrogenase type 2 (11HSD2) is minimal in human skin and ocular surface, permitting MR occupancy by excessive GCs that may induce deleterious effect, like in other non-classical MR-sensitive tissues (18). Our group has previously shown that topical MR blockade improves GCs-induced epidermal atrophy (19), wound re-epithelialization in GC-treated healthy human skin (20) and corneal re-epithelialization in GC-induced corneal wound healing delay (21). These observations show that deleterious effects of glucocorticoids on skin and corneal wound healing are MR-mediated. In addition, old studies identified that oral spironolactone (an MR blocking drug) was effective in the treatment (22) and prevention of skin rosacea (23).

### SUMMARY OF THE INVENTION

The present invention is defined by the claims. In particular, the present relates to methods for the treatment of ocular rosacea.

### DETAILED DESCRIPTION OF THE INVENTION

Here, the inventors show that the combination of mineralocorticoid receptor blockade in association with local ocular glucocorticoids that have high GR binding affinity, have superior effects as compared to MR blockade alone, without the side effects of glucocorticoids on corneal wound healing. In particular, they show that spironolactone, a MR antagonist, shifts the GR/MR balance in the cornea and ocular surface tissues, toward GR activation. The combination of MR antagonist and low dose of a GR activator further reduces corneal edema, corneal neovascularization and improves corneal wound healing. The combination of MR antagonist and triamcinolone that has a strong GR binding affinity reinforces the beneficial effects of MR antagonists. Finally, the inventors show that MR is overexpressed in ocular surface tissues and Meibomian glands of patients with ocular rosacea, and that transgenic rats that over express the human MR have molecular markers in their meibomian glands, similar to those of patients with OR. Thus, MR antagonists and GR agonist with strong GR affinity is a suitable combination for the treatment of OR.

### Main Definitions

As used herein, the term “ocular Rosacea” or “OR” has its general meaning in the art and refers to a chronic inflammatory and neurovascular diseases of the ocular surface and eyelids, associated with abnormal tear film lipids that can lead to corneal neovascularization, loss of transparency and ulceration.

As used herein the term “mineralocorticoid antagonist” or “MR antagonist” has its general meaning in the art. The MR antagonistic of a compound may be determined using various methods as described in J, Souque A, Wurtz J M, Moras D, Rafestin-Oblin M E. Mol Endocrinol. 2000 August; 14(8):1210-21; Fagart J, Seguin C, Pinon G M, Rafestin-Oblin M E. Mol Pharmacol. 2005 May; 67(5):1714-22 or Hellal-Levy C, Fagart J, Souque A, Wurtz J M, Moras D, Rafestin-Oblin M E. Mol Endocrinol. 2000 August; 14(8):1210-21. Typically, the transfection of the human mineralocorticoid receptor in COS cells together with a luciferase-expressing reporter gene allows to measure its transactivation activity in the presence of a candidate compound. In the context of the present invention, mineralocorticoid receptor antagonists are typically selective for the mineralocorticoid receptor as compared with the related receptors such as androgen receptor, estrogen receptors, glucocorticoid receptor, progesterone receptor, thyroid hormone receptors, peroxisome proliferator-activated receptors, retinoic acid receptor, farnesoid x receptor, pregnane x receptor, liver X receptor, vitamin D receptor, retinoid x receptor and the constitutive androstane receptor. By “selective” it is meant that the affinity of the antagonist for the mineralocorticoid receptor is at least 10-fold, typically 25-fold, more typically 100-fold, still typically 500-fold higher than the affinity for the related receptors. MR antagonists constitute a class of pharmacological compounds that are well known by the skilled artisan.

As used herein, the term “glucocorticoid receptor agonist” or “GR agonist” has its general meaning in the art and refers to a substance that interacts with a glucocorticoid receptor and enhances or increases a function of the glucocorticoid receptor. The term “glucocorticoid receptor agonist” encompasses both full and partial glucocorticoid receptor agonists. The term “glucocorticoid receptor agonist” encompasses selective modulators of the glucocorticoid receptor (SGRMs). SGRMs are known in the art, for example as described in Elmore, S. W., et al., J. Med. Chem. 44, 4481-4491; H. C. Owen, et al., Mol Cell Endocrinol 264 (2007), pp. 164-170 and De Bosscher K, et al., Proc Natl Acad Sci USA. 2005 Nov. 1; 102(44):15827-32. Typically, the GR binding affinity may be measured using a GR radiolabelled competitive binding assay such as described in Nehmé A, Lobenhofer E K, Stamer W D, Edelman J L. Glucocorticoids with different chemical structures but similar glucocorticoid receptor potency regulate subsets of common and unique genes in human trabecular meshwork cells. BMC Med Genomics. 2009; 2:58. Published 2009 Sep. 10. doi: 10.1186 1755-8794-2-58.). Briefly, fractions of IM-9 human B lymphoblast cell cytosol (300 μg proteins) are incubated for 6 h at 4° C. with 1.5 nM [3H]dexamethasone in the absence or presence of the compound to be tested in a buffer containing 10 mM Tris ethanesulfonic acid-NaOH (pH 7.4), 1 mM EDTA, 10 mM Na2MoO4, 20 mM β-mercaptoethanol and 10% glycerol. Nonspecific binding is determined in the presence of 10 μM triamcinolone. Following incubation, the samples are filtered rapidly under vacuum through glass fiber filters (GF/B, Packard) presoaked with 0.3% Poly (ethyleneimine) and rinsed several times with ice-cold 50 mM Tris-HCl using a 96-sample cell harvester (Unifilter, Packard). The filters are dried and counted for radioactivity in a scintillation counter (Topcount, Packard) using a scintillation cocktail (Microscint 0, Packard). The results are expressed as percent inhibition of the control radioligand specific binding. The IC₅₀ values (concentration causing half-maximal inhibition of control specific binding) and Hill coefficients (nH) were determined by non-linear regression analysis of the competition curves generated with mean replicate values using Hill equation curve fitting (Y=D+[(A−D)/(1+(C/C₅₀)^{nH})], where Y=specific binding, D=minimum specific binding, A=maximum specific binding, C=compound concentration, C₅₀=IC₅₀, and nH=slope factor). This analysis can be performed using the Hill software. The inhibition constants (Kᵢ) were calculated using the Cheng Prusoff equation (Kᵢ=IC₅₀/(1+(L/K_{D})), where L=concentration of radioligand in the assay, and K_{D}=affinity of the radioligand for the receptor). According to the present invention the standard reference compound is DEX, which is tested in each experiment at several concentrations to obtain a competition curve from which an IC₅₀ is calculated. Table 1 recapitulates the glucocorticoid receptor binding affinity for dexamethasone and triamcinolone acetonide.

TABLE 1
Glucocorticoid receptor binding affinity for dexamethasone and
triamcinolone acetonide determined according to Nehmé
A, Lobenhofer E K, Stamer W D, Edelman J L. Glucocorticoids
with different chemical structures but similar glucocorticoid
receptor potency regulate subsets of common and unique genes
in human trabecular meshwork cells. BMC Med Genomics. 2009; 2: 58.

| | IC₅₀ | Kᵢ | |
|---|---|---|---|
| Ligand | (nM) | (nM) | n_{H} |
| Dexamethasone | 5.36 ± 1.13 | 2.68 ± 0.58 | 1.06 ± 0.18 |
| Triamcinolone acetonide | 1.45 ± 0.50 | 0.72 ± 0.24 | 1.05 ± 0.07 |

As used herein, the term “GR agonist having an enhanced binding affinity” refers to any GR agonist having a lower IC₅₀ value than dexamethasone. Typically the IC₅₀ of the GR agonist having an enhanced binding affinity is 2; 2;5; 3; 3,5; 4; 4,5 times lower than the IC₅₀ of dexamethasone. The GR agonist having an enhanced binding affinity may specifically binds to GR (i.e ability to bind GR while having little or none affinity with other irrelevant molecules). Specificity can be exhibited by, e.g., an about 10:1, about 20:1, about 50:1, about 100:1, 10.000:1 or greater ratio of affinity in specific binding to GR versus non-specific binding to other irrelevant molecules.

As used herein, the term “combination” is intended to refer to all forms of administration that provide a first drug together with a further (second, third . . . ) drug. The drugs may be administered simultaneous, separate or sequential and in any order. Drugs administered in combination have biological activity in the subject to which the drugs are delivered.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of (such as corneal edema and opacity and corneal neovascularization), or ameliorate one or more symptoms (such as corneal ulcer, vision loss, and ocular pain) of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the expression “therapeutically effective amount” is meant a sufficient amount of a drug at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day.

### Methods of Treatment

The first object of the present invention relates to a method of treating ocular rosacea in a patient in need thereof comprising administering to the subject a therapeutically effective combination comprising at least one MR antagonist and at least one GR agonist having an enhanced binding affinity.

In particular embodiment, the present invention relates to a method of treating ocular rosacea in a patient in need thereof comprising administering to the subject a therapeutically effective combination comprising at least one MR antagonist and at least one GR agonist having an enhanced binding affinity wherein administration of the combination results in enhanced therapeutic efficacy and reduced side-effects relative to the administration of the MR antagonist alone or GR agonist alone.

In particular embodiment, the therapeutically effective combination comprising at least one MR antagonist and at least one GR agonist having an enhanced binding affinity is administered to a subject having ocular rosacea in order to prevent, cure, delay the onset of, reduce the severity of corneal neovascularisation and/or corneal edema and opacity and/or in order to ameliorate corneal re-epithelialization and/or corneal wound healing.

In particular embodiment, the administration of at least one MR antagonist and at least one GR agonist having an enhanced binding affinity to a subject having ocular rosacea ameliorates corneal re-epithelialization and/or corneal wound healing relative to administration of the MR antagonist alone or GR agonist alone.

Thus the invention refers to a method of promoting corneal re-epithelialization and/or corneal wound healing in a patient having ocular rosacea comprising administering to the subject a therapeutically effective combination comprising at least one MR antagonist and at least one GR agonist having an enhanced binding affinity.

A further object of the present invention relates to a method for enhancing the potency of a MR antagonist in a subject suffering from ocular rosacea as part of a treatment regimen, the method comprising administering to the subject a therapeutically effective amount of a GR agonist having an enhanced binding affinity.

A further object of the present invention relates to a method for reducing the side-effects of GR agonists in a subject suffering from ocular rosacea, as part of a treatment regimen, the method comprising administering to the subject a therapeutically effective combination comprising at least one MR antagonist and at least one GR agonist having an enhanced binding affinity.

In particular embodiment, the method of the invention is suitable to reduce corneal ulcer, corneal neovascularization, and/or corneal edema.

In particular embodiment, the method of the invention is suitable to delay corneal re-epithelialization and/or corneal wound healing.

In some embodiments, the mineralocorticoid receptor antagonists according to the invention generally are spironolactone-type steroidal compounds. The term “spironolactone-type” is intended to characterize a structure comprising a lactone moiety attached to a steroid nucleus, typically at the steroid “D” ring, through a spiro bond configuration. A subclass of spironolactone-type mineralocorticoid receptor antagonist compounds consists of epoxy-steroidal mineralocorticoid receptor antagonist compounds such as eplerenone. Another subclass of spironolactone-type antagonist compounds consists of non-epoxy-steroidal mineralocorticoid receptor antagonist compounds such as spironolactone.

The epoxy-steroidal mineralocorticoid receptor antagonist compounds used in the method of the present invention generally have a steroidal nucleus substituted with an epoxy-type moiety. The term “epoxy-type” moiety is intended to embrace any moiety characterized in having an oxygen atom as a bridge between two carbon atoms.

The term “steroidal,” as used in the phrase “epoxy-steroidal,” denotes a nucleus provided by a cyclopenteno-phenanthrene moiety, having the conventional “A”, “B”, “C”, and “D” rings. The epoxy-type moiety may be attached to the cyclopentenophenanthrene nucleus at any attachable or substitutable positions, that is, fused to one of the rings of the steroidal nucleus or the moiety may be substituted on a ring member of the ring system. The phrase “epoxy-steroidal” is intended to embrace a steroidal nucleus having one or a plurality of epoxy-type moieties attached thereto.

Epoxy-steroidal mineralocorticoid receptor antagonists suitable for use in the present methods include a family of compounds having an epoxy moiety fused to the “C” ring of the steroidal nucleus. Examples include 20-spiroxane compounds characterized by the presence of a 9α, 11α-substituted epoxy moiety, such as:
Pregn-4-ene-7,21-dicarboxylic acid, 9,11-epoxy-17-hydroxy-3-oxo-,γ-lactone, methyl ester, (7α, 11α, 17β)
Pregn-4-ene-7,21-dicarboxylic acid, 9,11-epoxy-17-hydroxy-3-oxo-, dimethyl ester, (7α, 11α, 17β)
3′ H-cyclopropa[6,7]pregna-4,6-diene-21-carboxylic acid,9,11-epoxy-6,7-dihydro-17-hydroxy-3-oxo-,γ-lactone, (6β, 7β, 11α, 17β)
Pregn-4-ene-7,21-dicarboxylic acid,9,11-epoxy-17-hydroxy-3-oxo-,7-(1-methylethyl) ester, monopotassium salt, (7α, 11α, 17β)
Pregn-4-ene-7,21-dicarboxylic acid,9,11-epoxy-17-hydroxy-3-oxo-,7-methylethyl) ester, monopotassium salt, (7α, 11α, 17β)
3′ H-cyclopropa[6,7]pregna-1,4,6-triene-21-carboxylic acid,9,11-epoxy-6,7-dihydro-17-hydroxy-3-oxo-,γ-lactone (6β, 7β, 11α)
3′ H-cyclopropa[6,7]pregna-4,6-diene-21-carboxylic acid,9,11-epoxy-6,7-dihydro-17-hydroxy-3-oxo-, methyl ester, (6β, 7β, 11α, 17β)
3′ H-cyclopropa [6,7] pregna-4,6-diene-21-carboxylic acid, 9,11-epoxy-6,7-dihydro-17-hydroxy-3-oxo-, monopotassium salt, (6β, 7β, 11α, 17β)
3′ H-cyclopropa[6,7]pregna-1,4,6-triene-21-carboxylic acid,9,11-epoxy-6,7-dihydro-17-hydroxy-3-oxo-,β-lactone (6β, 7β, 11α, 17β)
Pregn-4-ene-7,21-dicarboxylic acid,9,11-epoxy-17-hydroxy-3-oxo-,γ-lactone, ethyl ester, (7α, 11α, 17β)
Pregn-4-ene-7,21-dicarboxylic acid,9,11-epoxy-17-hydroxy-3-oxo-,γ-lactone, 1-methylethyl ester (7α, 11α, 17β)

A particular benefit of using epoxy-steroidal mineralocorticoid receptor antagonists, as exemplified by eplerenone, is the high selectivity of this group of mineralocorticoid receptor antagonists for the mineralocorticoid receptor. The superior selectivity of eplerenone results in a reduction in side effects that can be caused by mineralocorticoid receptor antagonists that exhibit non-selective binding to related receptors, such as androgen or progesterone receptors.

These epoxy steroids may be prepared by procedures described in Grob et al., U.S. Pat. No. 4,559,332. Additional processes for the preparation of 9,11-epoxy steroidal compounds and their salts are disclosed in Ng et al., WO97/21720 and Ng et al., WO98/25948.

Of particular interest is the compound eplerenone ((Pregn-4-ene-7,21-dicarboxylic acid, 9,11-epoxy-17-hydroxy-3-oxo-,γ-lactone, methyl ester, (7α, 11α, 17β)) (CAS No. 107724-20-9), also known as epoxymexrenone. Eplerenone is a mineralocorticoid receptor antagonist and has a higher selectivity for mineralocorticoid receptors than does, for example, spironolactone. Selection of eplerenone as the mineralocorticoid receptor antagonist in the present method would be beneficial to reduce certain side-effects such as gynecomastia that occur with use of mineralocorticoid receptor antagonists having less specificity.

Non-epoxy-steroidal mineralocorticoid receptor antagonists suitable for use in the present methods include a family of spirolactone-type compounds defined by Formula I:

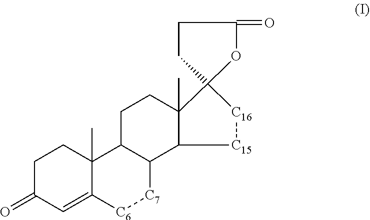

Wherein:

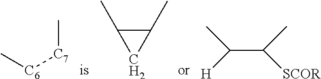

—R is lower alkyl of up to 5 carbon atoms, and

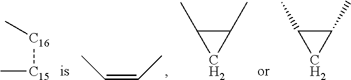

Lower alkyl residues include branched and unbranched groups, for example, methyl, ethyl and n-propyl.

Specific compounds of interest within Formula I are the following:
7α-acetylthio-3-oxo-4,15-androstadiene-[17(β-1′)-spiro-5′]perhydrofuran-2′-one;
3-oxo-7α-propionylthio-4,15-androstadiene-[17((β-1′)-spiro-5′]perhydrofuran-2′-one;
6β,7β-methylene-3-oxo4,15-androstadiene-[17((β-1′)-spiro-5′]perhydrofuran-2′-one;
15α,16α-methylene-3-oxo-4,7α-propionylthio-4-androstene[17(β-1′)-spiro-5′]perhydrofuran-2′-one;
6β,7β,15α,16α-dimethylene-3-oxo-4-androstene[17(β-1′)-spiro-5′]-perhydrofuran-2′-one;
7α-acetylthio-15β,16β-Methylene-3-oxo-4-androstene-[17(β-1′)-spiro-5′]perhydrofuran-2′-one;
15β,16β-methylene-3-oxo-7β-propionylthio-4-androstene-[17(β-1′)-spiro-5′]perhydrofuran-2′-one; and
6β,7β,15β,16β-dimethylene-3-oxo-4-androstene-[17(β-1′)-spiro-5′]perhydrofuran-2′-one.

Methods to make compounds of Formula I are described in U.S. Pat. No. 4,129,564 to Wiechart et al. issued on 12 Dec. 1978.

Another family of non-epoxy-steroidal compounds of interest is defined by Formula II:

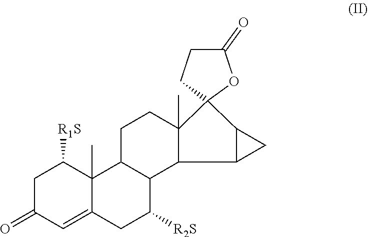

wherein R1 is C1-3-alkyl or C1-3 acyl and R2 is H or C1-3-alkyl.

Specific compounds of interest within Formula II are the following:
1α-acetylthio-15β,16β-methylene-7α-methylthio-3-oxo-17α-pregn-4-ene-21,17-carbolactone; and
15β,16β-methylene-1α,7α-dimethylthio-3-oxo-17α-pregn-4-ene-21,17-carbolactone.

Methods to make the compounds of Formula II are described in U.S. Pat. No. 4,789,668 to Nickisch et al. which issued 6 Dec. 1988.

Yet another family of non-epoxy-steroidal compounds of interest is defined by a structure of Formula III:

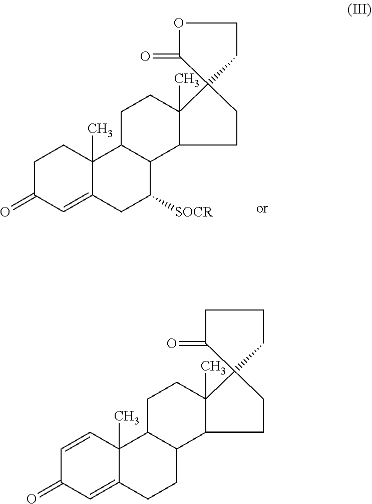

wherein R is lower alkyl, examples of which include lower alkyl groups of methyl, ethyl, propyl and butyl. Specific compounds of interest include:
3β,21-dihydroxy-17α-pregna-5,15-diene-17-carboxylic acid γ-lactone;
3β,21-dihydroxy-17α-pregna-5,15-diene-17-carboxylic acid γ-lactone 3-acetate;
3β,21-dihydroxy-17α-pregn-5-ene-17-carboxylic acid γ-lactone;
3β,21-dihydroxy-17α-pregn-5-ene-17-carboxylic acid γ-lactone 3-acetate;
21-hydroxy-3-oxo-17α-pregn-4-ene-17-carboxylic acid γ-lactone;
21-hydroxy-3-oxo-17α-pregna-4,6-diene-17-carboxylic acid γ-lactone;
21-hydroxy-3-oxo-17α-pregna-1,4-diene-17-carboxylic acid γ-lactone;
7α-acylthio-21-hydroxy-3-oxo-17α-pregn-4-ene-17-carboxylic acid γ-lactone; and
7α-acetylthio-21-hydroxy-3-oxo-17α-pregn-4-ene-17-carboxylic acid γ-lactone.

Methods to make the compounds of Formula III are described in U.S. Pat. No. 3,257,390 to Patchett which issued 21 Jun. 1966.

Still another family of non-epoxy-steroidal compounds of interest is represented by Formula IV:

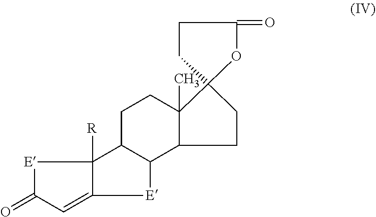

wherein E′ is selected from the group consisting of ethylene, vinylene and (lower alkanoyl) thioethylene radicals, E″ is selected from the group consisting of ethylene, vinylene, (lower alkanoyl) thioethylene and (lower alkanoyl) thiopropylene radicals; R is a methyl radical except when E′ and E″ are ethylene and (lower alkanoyl) thioethylene radicals, respectively, in which case R is selected from the group consisting of hydrogen and methyl radicals; and the selection of E′ and E″ is such that at least one (lower alkanoyl) thio radical is present.

One family of non-epoxy-steroidal compounds within Formula IV is represented by Formula V:

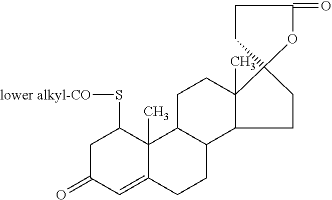

Another compound of Formula V is 1-acetylthio-17α-(2-carboxyethyl)-17β-hydroxy-androst-4-en-3-one lactone.

Another family of non-epoxy-steroidal compounds within Formula IV is represented by Formula VI:

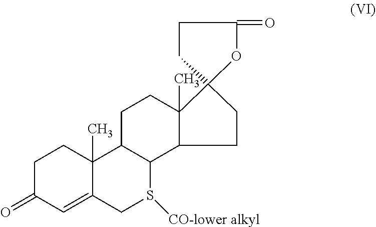

Exemplary compounds within Formula VI include the following:
7α-acetylthio-17α-(2-carboxyethyl)-17β-hydroxy-androst-4-en-3-one lactone;
7β-acetylthio-17α-(2-carboxyethyl)-17β-hydroxy-androst-4-en-3-one lactone;
1α,7α-diacetylthio-17α-(2-carboxyethyl)-17β-hydroxy-androsta-4,6-dien-3-one lactone;
7α-acetylthio-17αe-(2-carboxyethyl)-17β-hydroxy-androsta-1,4-dien-3-one lactone;
7α-acetylthio-17α-(2-carboxyethyl)-17β-hydroxy-19-norandrost-4-en-3-one lactone; and
7α-acetylthio-17α-(2-carboxyethyl)-17β-hydroxy-6α-methylandrost-4-en-3-one lactone.

In Formulae IV-VI, the term “alkyl” is intended to embrace linear and branched alkyl radicals containing one to about eight carbons. The term “(lower alkanoyl) thio” embraces radicals of the formula lower alkyl

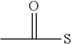

Of particular interest is the compound spironolactone (17-hydroxy-7α-mercapto-3-oxo-17α-pregn-4-ene-21-carboxylic acid γ-lactone acetate) having the following structure:

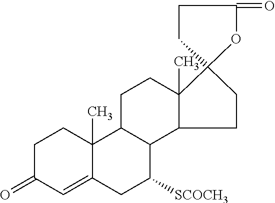

Methods to make compounds of Formulae IV-VI are described in U.S. Pat. No. 3,013,012 to Cella et al. which issued 12 Dec. 1961. Spironolactone is sold by G. D. Searle & Co., Skokie, Ill., under the trademark “ALDACTONE”, in tablet dosage form at doses of 25 mg, 50 mg and 100 mg per tablet.

Another family of steroidal mineralocorticoid receptor antagonists is exemplified by drospirenone, (6R-(6α,7α,8β,9α,10β,13β,14α,15α,16α,17β))-1,3′,4′,6,7,8,9,10,11,12,13,14,15,16,20,21-hexadecahydro-10,13-dimethylspiro [17H-dicyclopropa(6,7:15,16)cyclopenta(a)phenanthrene-17,2′ (5′ H)-furan)-3,5′ (2H)-dione, CAS registration number 67392-87-4. Methods to make and use drospirenone are described in patent GB 1550568 1979, priority DE 2652761 1976.

Crystalline forms that are easily handled, reproducible in form, easily prepared, stable, and which are non-hygroscopic have been identified for the mineralocorticoid receptor antagonist eplerenone. These include Form H, Form L, various crystalline solvates and amorphous eplerenone. These forms, methods to make these forms, and use of these forms in preparing compositions and medicaments, are disclosed in Barton et al., WO 01/41535 and Barton et al., WO 01/42272 both incorporated herein in their entirety.

Mineralocorticoid receptor antagonists according to the invention may also be non-steroidal. For example, classes of non-steroidal MR antagonists have just begun to emerge over the past few years (Meyers, Marvin J1; Hu, Xiao Expert Opinion on Therapeutic Patents, Volume 17, Number 1, January 2007, pp. 17-23 (7) and Piotrowski DW. Mineralocorticoid Receptor Antagonists for the Treatment of Hypertension and Diabetic NephropathyJ. Med. Chem. 2012, 55, 7957-7966). For instance, dihydropyrymidines have been shown to display MR antagonism (Activation of Mineralocorticoid Receptors by Exogenous Glucocorticoids and the Development of Cardiovascular Inflammatory Responses in Adrenalectomized Rats. Young M J, Morgan J, Brolin K, Fuller P J, Funder J W. Endocrinology. 2010 Apr. 21). Furthermore, Arhancet el al. disclose other class of non-steroidal MR antagonists (Arhancet G B, Woodard S S, Dietz J D, Garland D J, Wagner G M, Iyanar K, Collins J T, Blinn J R, Numann R E, Hu X, Huang H C. Stereochemical Requirements for the Mineralocorticoid Receptor Antagonist Activity of Dihydropyridines. J Med Chem. 2010 Apr. 21). Other exemplary non-steroidal mineralocorticoid receptor antagonists include but are not limited to those described in US 20090163472 WO2004052847, WO 2008053300 WO2008104306, WO2007025604, WO201264631, WO2008126831, WO2012008435, WO2010104721, WO200985584, WO200978934, WO2008118319, WO200917190, WO200789034, WO2012022121, WO2012022120, WO2011141848 and WO200777961 that are hereby incorporated by reference into the present disclosure.

In some embodiments, the mineralocorticoid receptor antagonist is selected from the group consisting of:

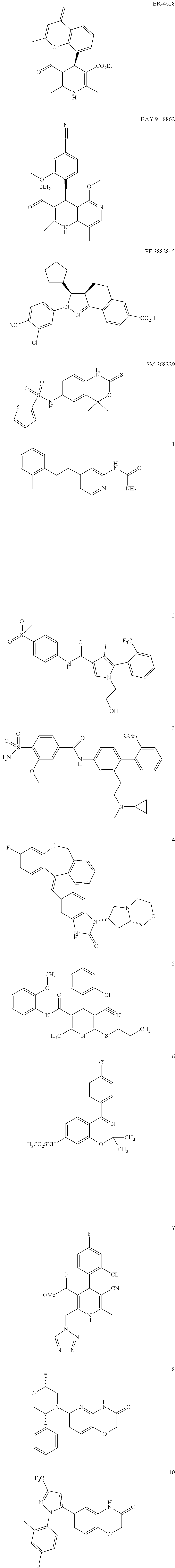

In some embodiments, the MR antagonist of the present invention is finerenone (Liu LC, Schutte E, Gansevoort R T, van der Meer P, Voors A A. Finerenone: third-generation mineralocorticoid receptor antagonist for the treatment of heart failure and diabetic kidney disease. Expert Opin Investig Drugs. 2015; 24(8):1123-35) which has the formula of:

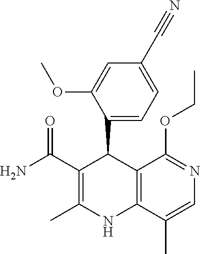 [SMILES: CCOc1ncc(C)c2c1[C@H](c1ccc(C#N)cc1OC)C(C(N)=O)=C(C)N2]

In some embodiments, the MR antagonist of the present invention is selected from the group consisting of finerenone, spironolactone, canrenone, potassium canrenoate and eplerenone.

In some embodiments, the MR antagonist is an inhibitor of MR expression. An “inhibitor of expression” refers to a natural or synthetic compound that has a biological effect to inhibit the expression of a gene. In some embodiments, said inhibitor of gene expression is a siRNA, an antisense oligonucleotide or a ribozyme. For example, anti-sense oligonucleotides, including anti-sense RNA molecules and anti-sense DNA molecules, would act to directly block the translation of MR mRNA by binding thereto and thus preventing protein translation or increasing mRNA degradation, thus decreasing the level of MR, and thus activity, in a cell. For example, antisense oligonucleotides of at least about 15 bases and complementary to unique regions of the mRNA transcript sequence encoding MR can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of expression for use in the present invention. MR gene expression can be reduced by contacting a subject or cell with a small double stranded RNA (dsRNA), or a vector or construct causing the production of a small double stranded RNA, such that MR gene expression is specifically inhibited (i.e. RNA interference or RNAi). Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically cells expressing MR. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art. Non-viral vectors can be used such as cationic lipids, liposomes, particulate polymeric systems, nucleic acid particles, dendrimers, cationic peptides such as VP22 or other parts of virus peptides, electric field such as electrotransfer but also ultrasounds, guns, corticotransfection. The antisense oligonucleotide or siRNA could also be used naked as drops of peri-ocular injections in solutions

Thus, in some embodiments, MR antagonists are antisense oligonucleotide, siRNA or shRNA.

In some embodiments, the GR agonist having an enhanced binding affinity is selected from the group consisting of:
triamcinolone acetonide,
RU 24782, RU 24858, RU 40066, and RU28362 (J Neuroendocrinol 2006 February; 18(2):129-38. doi: 10.1111/j.1365-2826.2005.01396.x) that are steroid GR agonists
11-ketosteroid 11-ketodexamethasone is a glucocorticoid receptor agonist [Mol Cell Endocrinol 2004 Feb. 12; 214(1-2):27-37. doi: 10.1016/j.mce.2003.11.027]
LGD-5552
ZK 216348 (Bayer Schering Pharma AG)
SA22465 and its active metabolites such as SA22313
AL-438
ZK 216348, and
BI 115

In some embodiments, the GR agonist of the present invention is selected from tetrahydronaphthaline-methylbenzoxazinones as described in WO2006/000398 and WO2006/000401) each member of which has a binding below 100 nM to GR.

In some embodiments, the GR agonist of the present invention is selected from natural products such as peppermint oil, L-limonene and L-menthol [2021 Apr. 29; 22(9):4747. doi: 10.3390/ijms22094747.

In some embodiments, the GR agonist is selective GR agonist (SEGRA) as described in Schäcke H, Berger M, Rehwinkel H, Asadullah K. Selective glucocorticoid receptor agonists (SEGRAs): novel ligands with an improved therapeutic index. Mol Cell Endocrinol. 2007 Sep. 15; 275(1-2):109-17.

In some embodiments, the GR agonists results from the translation of specific mRNA encoding the GR or self-amplifying mRNA encoding the GR or from plasmid DNA encoding the GR.

In some embodiments, the MR antagonist and the GR agonist of the present invention are administered to the patient separately.

In some embodiments, the MR antagonist and the GR agonist of the present invention are administered to the patient in the same pharmaceutical composition.

Therefore, a further object of the present invention relates to a pharmaceutical composition comprising a combined amount of at least one MR antagonist and at least one GR agonist having an enhanced binding affinity.

Therefore, a further object of the present invention relates to a pharmaceutical composition comprising a combined amount of at least one MR antagonist and at least one GR agonist having an enhanced binding affinity for use in a method of treating ocular rosacea in a patient in need thereof.

The MR antagonist and the GR agonist of the present invention are preferably administered locally (topical) or systemically or using loco regional administrations (sub conjunctival, sub tenon, peri bulbar, latero bulbar retro bulbar, sub tenon injections or delivery). Preferably, the MR and the GR agonist are administered topically. For instance, topical administration is carried out by means known to one of ordinary skill in the art and that typically include passive diffusion, iontophoresis, sonophoresis, electroporation, mechanical pressure, osmotic pressure gradient, occlusive cure, microinjections, by needle-free injections by means of pressure, by microelectric patches, or any combination thereof.

In some embodiments, the MR antagonist and the GR agonist of the present invention are administered to the patient via a subconjunctival injection. As used herein, the term “subconjunctival injection” refers to a type of periocular route of injection for ocular drug administration by administration of a medication either under the conjunctiva or underneath the conjunctiva lining the eyelid. Using the subconjunctival injection bypasses the fatty layers of the bulbous conjunctiva and putting medications adjacent to sclera that is permeable to water, this will increase the penetration of the water-soluble drug into the eye. Any type of suspension, polymeric implants, particulate systems made of polymers or of lipids or of composition of polymers with lipids could be delivered subconjunctivally.

In some embodiments, the MR antagonist of the present invention is administered to the subject in a topical pharmaceutical composition. The topical pharmaceutical composition may be in liquid, pasty or solid form, and more particularly in the form of emulsions (either oil-in-water or water-in-oil emulsions), such as creams or lotions; micro emulsions; gels; ointments; liposomes; powders; aqueous solutions or suspensions, such as standard ophthalmic preparations; aerosols; sprays; and washes. It may also be in the form of a suspension of microspheres or nanospheres or nanomicelles made of lipid or polymer or both or a polymer patch and a hydrogel allowing controlled release. This pharmaceutical composition for topical application may be in anhydrous form, in aqueous form or in the form of an emulsion or nanomicelles. In some embodiments, the pharmaceutical composition for topical application is in the form of a solution, a gel or an emulsion.

When the pharmaceutical composition according to the invention is in the form of an emulsion, it comprises at least one surfactant. An emulsion comprises a mixture of two immiscible liquids, one of which is dispersed in the other in the form of fine droplets (micelles or nanomicelles); the dispersion is stabilized owing to the action of surfactants that modify the structure and the ratio of forces at the interface, and therefore increase the stability of the dispersion by decreasing the interfacial tension energy. The surfactant may be ionic (anionic, cationic or amphoteric), or nonionic. Among the surfactants that can be used according to the invention, examples that may be mentioned include: glyceryl/PEG100 stearate sold under the name Arlacel 165FL by the company Uniqema or under the name Simulsol 165 by the company SEPPIC, polyoxyethylenated fatty acid esters such as Arlatone 983 from the company Uniqema or the polyoxyethylenated stearyl alcohol (2) sold under the name Brij72 combined with the polyoxyethylenated stearyl alcohol (21) sold under the name Brij721 by the company Uniqema, sorbitan esters such as sorbitan oleate sold under the name Arlacel 80 by the company ICI or sold under the name Crill 4 by the company Croda, sorbitan sesquioleate sold under the name Arlacel 83 by the company ICI or sold under the name Montane 83 by the company SEPPIC, or else sorbitan isostearate; or else ethers of fatty alcohols.

Examples of cationic emulsions are the emulsions disclosed in WO93/18852, i.e. oil/water type emulsion which comprises colloid particles having an oily core surrounded by an interfacial film, the film comprising surface active agents, lipids or both, said emulsions being characterised in that at least part of the surface active agents or lipids in the interfacial film have positively charged polar groups and further in that the colloid particles have a positive zeta potential. The interfacial film may also comprise non-ionic surfactants or lipids. In some embodiments, the emulsion consists of the emulsion described in WO2006003519 and thus comprises comprises (expressed in % w/w): 0.5-20% oily carrier, preferably 0.5-10%; 0.01-2% cationic surfactants or lipids, preferably 0.02-0.4% and optionally a non-ionic surfactant in a range of 0.05-3%, preferably in a range of 0.1-2%.

Examples of anionic emulsions are the emulsions described in Klang, S et al. 2000. Influence of emulsion droplet surface charge on indomethacln ocular tissue distribution. Pharm Dev Technol 5(4): p. 521-32 and Abdulrazik, M, et al. 2001. Ocular delivery of cyclosporin A. II. Effect of submicron emulsion's surface charge on ocular distribution of topical cyclosporin A.

STP Pharma Sciences 11(6): p. 427-432., i.e. oil/water type emulsion which comprises colloid particles having an oily core surrounded by an interfacial film, the film comprising surface active agents, lipids or both, said emulsions being characterised in that at least part of the surface active agents or lipids in the interfacial film have negatively charged polar groups and further in that the colloid particles have a negative zeta potential. The interfacial film may also comprise non-ionic surfactants or lipids.

In some embodiments, the topical pharmaceutical composition comprises microspheres which can release drug loads over various time periods. These microspheres, which when inserted into the subconjunctival (such as a sub-tenon) space or into the vitreous of an eye provide therapeutic levels of a MR antagonist and/or GR agonist, for extended periods of time (e.g., for about one week or more). As used herein the term “microsphere” has its general meaning in the art and refers to a small diameter or dimension device or element that is structured, sized, or otherwise configured to be administered subconjunctivally (i.e. sub-tenon). Microspheres or microparticles includes particles, micro or nanospheres, small fragments, microparticles, nanoparticles, fine powders and the like comprising a biocompatible matrix encapsulating or incorporating a therapeutic agent. Microspheres are generally biocompatible with physiological conditions of an eye and do not cause significant adverse side effects. Microspheres administered intraocular can be used safely without disrupting vision of the eye. Microspheres have a maximum dimension, such as diameter or length, less than 1 mm.

Suitable polymeric materials for use in the topical pharmaceutical composition include materials which are compatible (i.e. biocompatible) with the eye so as to cause no substantial interference with the functioning or physiology of the eye. Such materials preferably are at least partially and more preferably substantially completely biodegradable polymer. “Biodegradable polymer” means a polymer or polymers which degrade in vivo, and wherein erosion of the polymer or polymers over time occurs concurrent with or subsequent to release of the therapeutic agent. The terms “biodegradable” and “bioerodible” are equivalent and are used interchangeably herein. A biodegradable polymer may be a homopolymer, a copolymer, or a polymer comprising more than two different polymeric units. Examples of useful polymeric materials include, without limitation, such materials derived from and/or including organic esters and organic ethers, which when degraded result in physiologically acceptable degradation products, including the monomers. Also, polymeric materials derived from and/or including, anhydrides, amides, orthoesters and the like, by themselves or in combination with other monomers, may also find use. The polymeric materials may be addition or condensation polymers, advantageously condensation polymers. The polymeric materials may be cross-linked or non-cross-linked, for example not more than lightly cross-linked, such as less than about 5%, or less than about 1% of the polymeric material being cross-linked. For the most part, besides carbon and hydrogen, the polymers will include at least one of oxygen and nitrogen, advantageously oxygen. The oxygen may be present as oxy, e.g. hydroxy or ether, carbonyl, e.g. non-oxo-carbonyl, such as carboxylic acid ester, and the like. The nitrogen may be present as amide, cyano and amino. The polymers set forth in Heller, Biodegradable Polymers in Controlled Drug Delivery, In: CRC Critical Reviews in Therapeutic Drug Carrier Systems, Vol. 1, CRC Press, Boca Raton, Fla. 1987, pp 39-90, which describes encapsulation for controlled drug delivery, may find use in the present microspheres. Of particular interest are polymers of hydroxyaliphatic carboxylic acids, either homo- or copolymers, and polysaccharides. Included among the polyesters of interest are homo- or copolymers of D-lactic acid, L-lactic acid, racemic lactic acid, glycolic acid, caprolactone, and combinations thereof. Copolymers of glycolic and lactic acid are of particular interest, where the rate of biodegradation is controlled by the ratio of glycolic to lactic acid. The percent of each monomer in poly(lactic-co-glycolic)acid (PLGA) copolymer may be 0-100%, about 15-85%, about 25-75%, or about 35-65%. In some embodiments, 25/75 PLGA and/or 50/50 PLGA copolymers are used. In some embodiments, the polymer is a cyclodextrin polymer. In some embodiments, the cyclodextrin is selected from the group consisting of alpha-cyclodextrin, beta-cyclodextrin, and gamma-cyclodextrin. In some embodiments, the polymer is a water-soluble polymer, which can be at least one selected from the group consisting of alginic acid, calcium carboxymethyl cellulose, sodium carboxymethyl cellulose, Carbomer, carrageenan, chitosan, guar gum, hypromellose, polyvinyl alcohol, polyethylene oxide, polyvinyl pyrrolidone, hydroxypropyl cellulose, hydroxypropyl ethyl cellulose, vinyl pyrrolidone-vinyl acetate copolymer and

Eudragit, among which polyvinyl pyrrolidone. In some embodiments, the comprises one or more of an alkyl substituted polylactide or/and a polymer prepared by melt polycondensation of one or more substituted or unsubstituted C6-Cs 2-hydroxyalkyl acid(s) as described in WO2019145430. In particular, the composition results from the spontaneous encapsulation of the MR antagonist/GR agonist within very small micellar structures formed from the co-polymers of the 2-hydroxyalkyl acid(s) with mPEG, and thus consists of a clear aqueous formulation may be prepared. Additionally, the hydrophilic shells of such micellar structures may advantageously interact intimately with naturally-hydrated tissue surfaces. Even more advantageously, the greatly enhanced surface area of drug-loaded micellar structures facilitates rapid and efficient transfer of drug into the tissue onto which the formulation is administered.

In some embodiments, the ophthalmic compositions of the present invention comprises hydrogenated phospholipids (HPL) structured as liposomes. The so obtained formulations containing complexes of phospholipids (in form of liposomes) with sodium salt of lactobionic acid are characterized by increased ability to deliver the active principle; due to the reduced surface tension and the known capacity of liposomes to interact with ocular surfaces, also the formulation stability is increased with consequent improved delivery and bioavailability of the active principle.

In some embodiments, the MR antagonist/GR agonist is administered via a medical device. In some embodiments, the medical device consists of an implant. The implants are typically solid, and may be formed as particles, sheets, patches, plaques, films, discs, fibers, rods, and the like, or may be of any size or shape compatible with the selected site of implantation, as long as the implants have the desired release kinetics and deliver an amount of active agent that is therapeutic for the intended medical condition of the eye. The implant may be a sustained release device or a sustained release-system that dissolves over time. In some embodiments, the sustained-release drug delivery system includes a polymer. In some embodiments, the polymer may be configured so that it dissolves over time (such as e.g. for up to 6 months) when implanted into the eye. In certain embodiments, the polymer may comprise a polylactic-coglycolic acid (PLGA). In a specific embodiment, the polymer may comprise poly (D,L-lactide-co-glycolide) PLGA. Suitable sustained-release device are configured such that a pharmaceutically acceptable amount of the drug is released daily. Exemplary suitable sustained release devices are disclosed, for example, in U.S. Pat. Nos. 5,378,475; 5,773,019; 6,217,895; 6,375,972; 6,548,078; 8,252,307; 8,574,659 and 8,871,241, the disclosures of which are incorporated by reference. In some embodiments, the device is a contact lens delivery system that releases medication over an extended period. The lens generally only lasts for a matter of hours or days before dissolving or releasing all of the therapeutic compound. In some embodiments, the device is palpebral patch.

The invention will be further illustrated by the following figures and examples. However, these examples and figures should not be interpreted in any way as limiting the scope of the present invention.

### FIGURES

FIG. 1. Corneal de-epthelialization and limbal resection model treatment schemes.

FIG. 2. Spironolactone up-regulates GR and the GR/MR balance in favor of GR pathway activation.

FIG. 2: MR specific mineralocorticoid (MR) antagonist significantly reduces corneal thickness, edema and neovessels. MR antagonism using spironolactone (SPL) administered systemically reduces the corneal edema, corneal thickness (A-B, measure by corneal thickness in vivo using coherence optical tomography) and corneal neovascularization (C) induced by limbal deficiency. (C) The anti-angiogenic effect of MR antagonism is further confirmed by eplerenone (Eple, B).

FIG. 3. Combined treatment with spironolactone (SPL) and triamcinolone acetonide (TA) reduced infiltration of ED1 and IBA1-positive inflammatory cells. (A) Immunohistochemistry labelling of macrophages/microglia using IBA1 and infiltrating cells using ED1 on corneal sections at day 3. (B) Quantification of the surface of cornea positive for IBA1- or ED1-positive cells. Spironolactone also reduces infiltration of IBA1-positive macrophages and ED1 positive inflammatory cells in corneal lesions. Spironolactone+ TA acts synergistically on the infiltrating IBA1- or ED1-positive cells, with significantly better anti-inflammatory effect then spironolactone or TA alone.

FIG. 4. Spironolactone up-regulates GR and the GR/MR balance in favor of GR pathway activation. The corneal de-epithelialization and limbal deficiency model induces a down regulation of GR genes at day 3 and 7, and is restored at day 14 when the cornea is healed. MR expression is down regulated at day 3, upregulated at day 7 and normalized at Day 14. Treatment with spironolactone tilts the MR/GR balance in favour of GR at day 7. Spironolactone normalizes the GR/MR balance, favoring GR pathway activation.

FIG. 5. Combined treatment with spironolactone (SPL) and triamcinolone acetonide (TA) potentiates the anti-angiogenic effect of and improves delayed wound healing and favor healing of corneal ulcer and transparency. Co-administration of spironolactone and local TA has an additive effect on corneal angiogenesis (such as reducing (A) corneal neovascularization and (B) corneal ulcer).

### EXAMPLE

### MR Expression is Increased in Ocular Tissues of Patients With OR

In human ocular surface tissues, GR and MR are both expressed in the epidermis, hair follicles, sebaceous glands, meibomian glands and conjunctiva. In patients with OR, MR immunostaining is enhanced, particularly in the epidermis, meibomian glands and conjunctiva (data not shown).

### Corneal Neovascularization, Corneal Edema, Corneal Inflammation and Corneal Ulcer Induced by Limbal Deficiency is Alleviated by MR Blockade

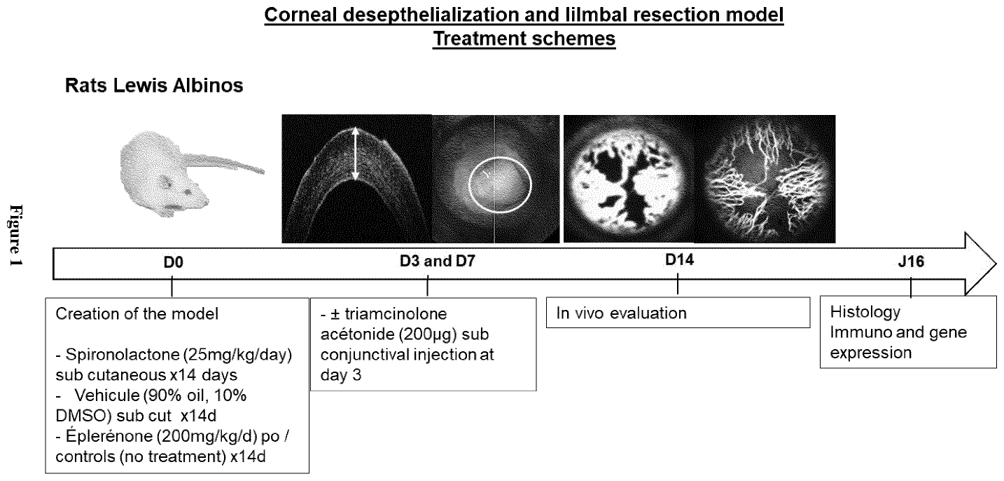

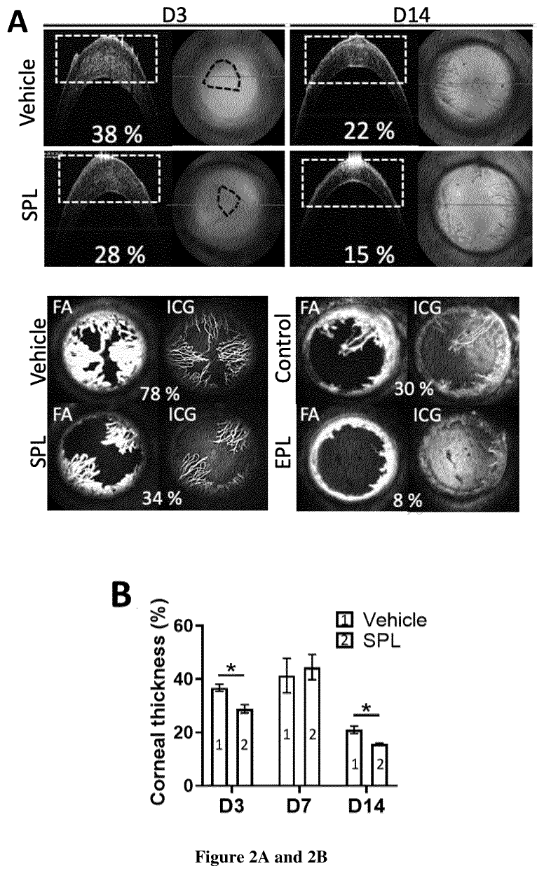

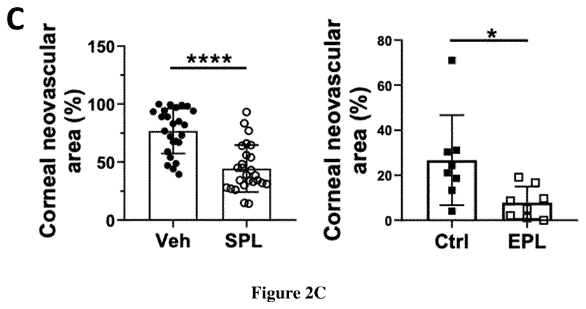

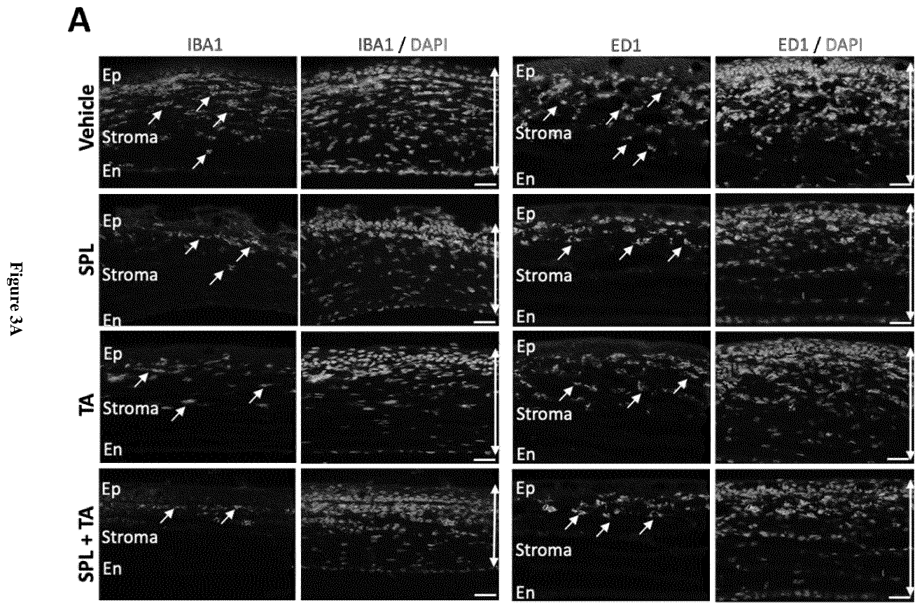

Corneal neovascularization (CN) is a severe vision-threatening complication of OR. We previously showed that invalidation of MR in endothelial cells prevents corneal neovascularization induced by limbal deficiency (24). In a rat model of CN induced by de-epithelialization and limbal resection (FIG. 1), MR antagonists (spironolactone and eplerenone administered systemically) decrease corneal edema, corneal thickness (FIG. 2A-B) inhibited corneal neovascularization (FIG. 2A-C) and improved corneal re-epithelialization (data not shown). This data shows that not only spironolactone, but also eplerenone, which is highly specific of MR, had significant beneficial effect on the regression of corneal angiogenesis, demonstrating that the effect is MR-mediated.

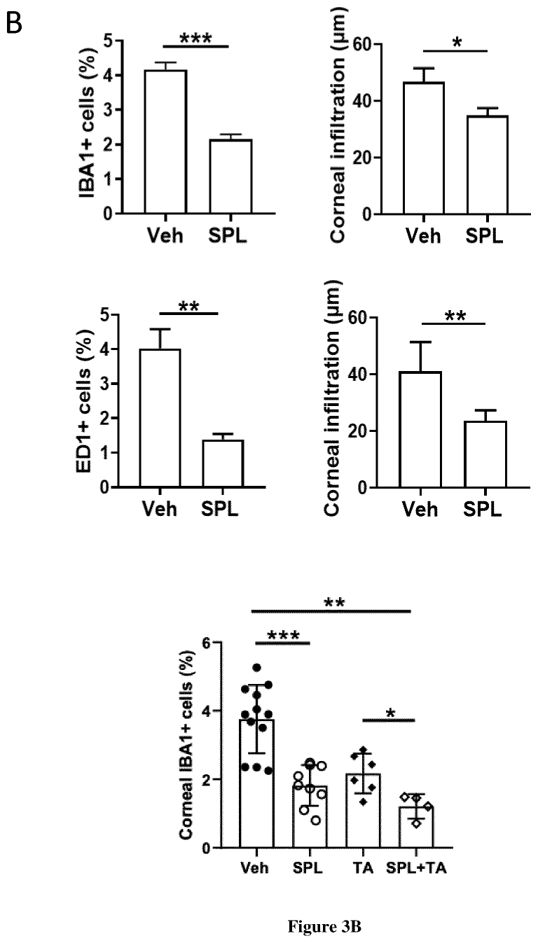

Spironolactone also reduced infiltration of IBA1-positive macrophages and ED1 positive inflammatory cells in corneal lesions (FIG. 3A-B). Spironolactone is at least as efficient as TA on ED1- and IBA1-cells infiltration. However, the combination spironolactone and TA acts synergistically reduced infiltration of IBA1-positive cells, with significantly better anti-inflammatory effect then spironolactone or TA alone (FIG. 3A-B). On ED1 positive cells infiltration, the combination spironolactone and TA is more efficient than TA alone or spironolactone alone (FIG. 3A).

### MR Antagonists Increases the GR/MR Balance in Ocular Surface Tissues

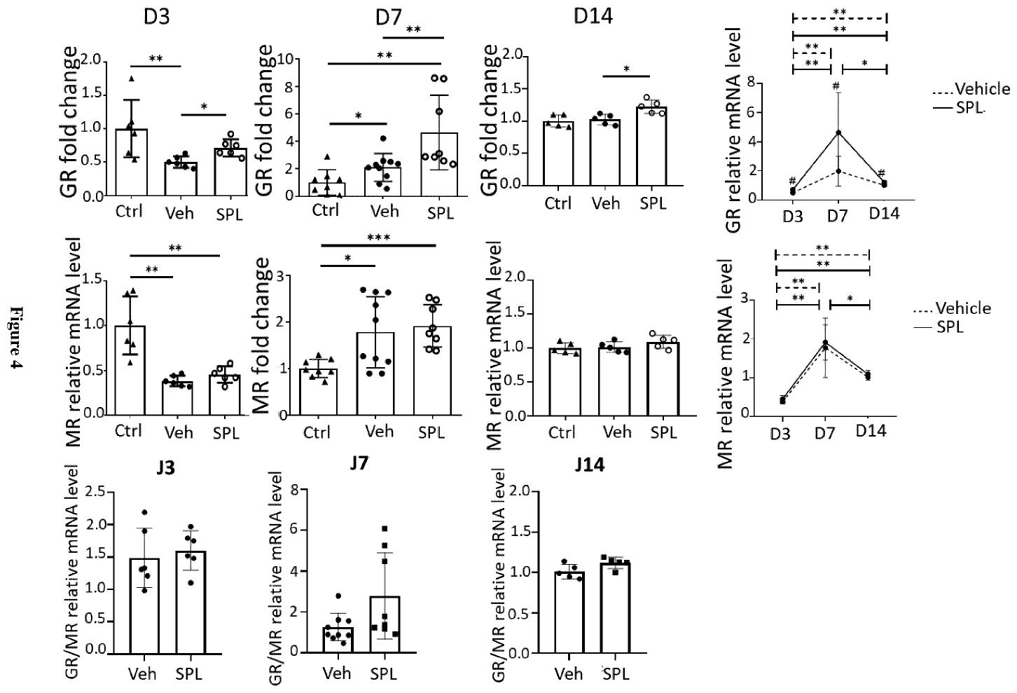

MR antagonism up-regulated the GR expression in the cornea/limbus of rats with limbal deficiency, tilting the GR/MR balance in favor of anti-inflammatory and anti-angiogenic GR pathway (FIG. 4). GR expression in the cornea is down-regulated in the model of limbal deficiency at day 3 and 7 and is restored at day 14 when the cornea has healed (FIG. 4). Spironolactone up regulates and normalizes the GR expression at day 3 and 7 (FIG. 4). MR expression is down regulated at day 3, upregulated at day 7 and normalized at Day 14 (FIG. 4). Spironolactone does not influence MR expression. Thus, spironolactone normalizes the GR/MR balance, favoring GR pathway activation. This result suggests that spironolactone effect could result from a shift toward GR pathway activation in the cornea and ocular surface tissues.

### Co-Administration of Specific GR Agonist+MR Antagonist is Superior to MR Antagonist Alone

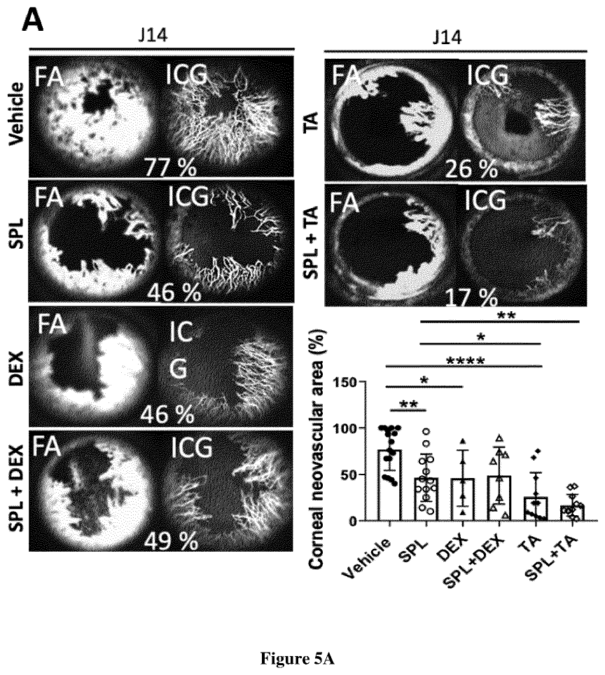

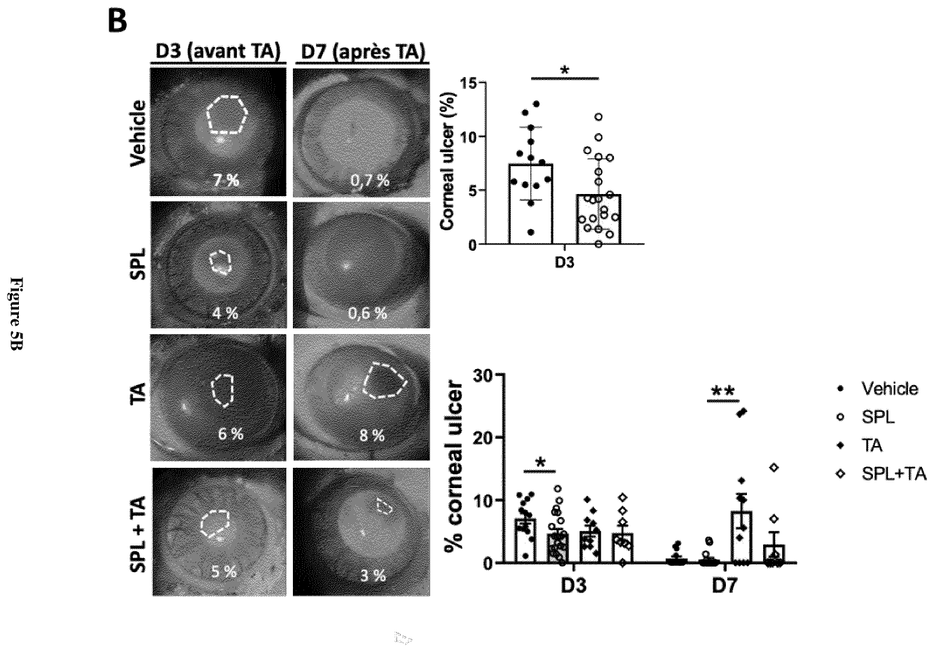

Spironolactone, dexamethasone and TA have similar anti-angiogenic effects or corneal limbal deficiency model (FIG. 5A-5B). Co-administration of triamcinolone acetonide (TA), a highly specific GR activator with spironolactone almost completely inhibited the CN suggesting that restoration of GR activation over MR carries therapeutic potential (FIG. 5A). Moreover, spironolactone improved the corneal re-epithelialization (i.e corneal wound) delayed by the TA injection (FIG. 5B). Co-administration of MR antagonist and local dexamethasone did not improve the effect of spironolactone or dexamethasone on corneal neovascularization and corneal edema (FIG. 5A). This data shows that the combination of spironolactone with TA, not only favors the corneal transparency by inducing a better regression of neovessels, but also protects from wound healing delay, induced by glucocorticoids.

### Co-Administration of Specific GR Agonist+MR Antagonist Induces the Regulation of Specific Genes

A full transcriptomic regulation induced in the cornea and limbus at 3 and 7 days after spironolactone systemic injection alone or with TA has been realized. 75 genes are up or down regulated by spironolactone alone, but 285 genes are significantly regulated by the co-administration of spironolactone and TA (data not shown). 172 genes are specific to the combination as compared to TA alone or to spironolactone alone (data not shown).

This result demonstrates that the combination of spironolactone and TA induces the regulation of specific genes that are different from the genes regulated by TA or spironolactone alone. The combination of MR antagonist and a specific GR agonist, such TA, is thus exerting specific effects and synergistic effects as compared to TA or spironolactone alone. These effects are different from the effect of specific GR agonist alone or spironolactone alone. The combination is thus a novel therapeutic entity.

### REFERENCES

Throughout this application, various references describe the state of the art to which this invention pertains. The disclosures of these references are hereby incorporated by reference into the present disclosure.
1. Tan J, Schöfer H, Araviiskaia E, Audibert F, Kerrouche N, Berg M, et al. Prevalence of rosacea in the general population of Germany and Russia—The RISE study. J Eur Acad Dermatol Venereol JEADV. mars 2016;30(3):428-34.
2. Vieira AC, Mannis MJ. Ocular rosacea: Common and commonly missed. J Am Acad Dermatol. 1 déc 2013;69(6, Supplement 1):S36-41.
3. Steinhoff M, Schauber J, Leyden JJ. New insights into rosacea pathophysiology: a review of recent findings. J Am Acad Dermatol. déc 2013;69(6 Suppl 1):S15-26.
4. Peters EMJ, Ericson ME, Hosoi J, Seiffert K, Hordinsky MK, Ansel JC, et al. Neuropeptide control mechanisms in cutaneous biology: physiological and clinical significance. J Invest Dermatol. sept 2006;126(9):1937-47.
5. Zouboulis CC. The skin as an endocrine organ. Dermatoendocrinol. 2009;1(5):250-2.
6. Gerber PA, Buhren BA, Steinhoff M, Homey B. Rosacea: The cytokine and chemokine network. J Investig Dermatol Symp Proc. déc 2011;15(1):40-7.
7. Machalińska A, Zakrzewska A, Markowska A, Safranow K, Wiszniewska B, Parafiniuk M, et al. Morphological and Functional Evaluation of Meibomian Gland Dysfunction in Rosacea Patients. Curr Eye Res. 2016;41(8):1029-34.
8. Buddenkotte J, Steinhoff M. Recent advances in understanding and managing rosacea. F1000Research. 2018;7.
9. Cox SM, Nichols JJ. The neurobiology of the meibomian glands. Ocul Surf. juill 2014;12(3):167-77.
10. Yamasaki K, Kanada K, Macleod DT, Borkowski AW, Morizane S, Nakatsuji T, et al. TLR2 expression is increased in rosacea and stimulates enhanced serine protease production by keratinocytes. J Invest Dermatol. mars 2011;131(3):688-97.
11. Sulk M, Seeliger S, Aubert J, Schwab VD, Cevikbas F, Rivier M, et al. Distribution and expression of non-neuronal transient receptor potential (TRPV) ion channels in rosacea. J Invest Dermatol. avr 2012;132(4):1253-62.
12. Moore C, Cevikbas F, Pasolli HA, Chen Y, Kong W, Kempkes C, et al. UVB radiation generates sunburn pain and affects skin by activating epidermal TRPV4 ion channels and triggering endothelin-1 signaling. Proc Natl Acad Sci U S A. 20 août 2013;110(34):E3225-3234.
13. Buhl T, Sulk M, Nowak P, Buddenkotte J, McDonald I, Aubert J, et al. Molecular and Morphological Characterization of Inflammatory Infiltrate in Rosacea Reveals Activation of Th1/Th17 Pathways. J Invest Dermatol. sept 2015;135(9):2198-208.
14. Wladis EJ, Adam AP. Treatment of ocular rosacea. Surv Ophthalmol. juin 2018;63(3):340-6.
15. Slominski AT, Manna PR, Tuckey RC. Cutaneous glucocorticosteroidogenesis: securing local homeostasis and the skin integrity. Exp Dermatol. juin 2014;23(6):369-74.
16. Zmijewski MA, Sharma RK, Slominski AT. Expression of molecular equivalent of hypothalamic-pituitary-adrenal axis in adult retinal pigment epithelium. J Endocrinol. avr 2007;193(1):157-69.
17. Bhat YJ, Manzoor S, Qayoom S. Steroid-induced rosacea: a clinical study of 200 patients. Indian J Dermatol. janv 2011;56(1):30-2.
18. Jaisser F, Farman N. Emerging Roles of the Mineralocorticoid Receptor in Pathology: Toward New Paradigms in Clinical Pharmacology. Pharmacol Rev. janv 2016;68(1):49-75.
19. Maubec E, Laouénan C, Deschamps L, Nguyen VT, Scheer-Senyarich I, Wackenheim-Jacobs A-C, et al. Topical Mineralocorticoid Receptor Blockade Limits Glucocorticoid-Induced Epidermal Atrophy in Human Skin. J Invest Dermatol. juill 2015;135(7):1781-9.
20. Nguyen VT, Farman N, Maubec E, Nassar D, Desposito D, Waeckel L, et al. Re-Epithelialization of Pathological Cutaneous Wounds Is Improved by Local Mineralocorticoid Receptor Antagonism. J Invest Dermatol. 2016;136(10):2080-9.
21. Dahmana N, Mugnier T, Gabriel D, Kaltsatos V, Bertaim T, Behar-Cohen F, et al. Topical Administration of Spironolactone-Loaded Nanomicelles Prevents Glucocorticoid-Induced Delayed Corneal Wound Healing in Rabbits. Mol Pharm. 5 mars 2018;15(3):1192-202.
22. Aizawa H, Niimura M. Oral spironolactone therapy in male patients with rosacea. J Dermatol. mai 1992;19(5):293-7.
23. Spoendlin J, Voegel JJ, Jick SS, Meier CR. Spironolactone may reduce the risk of incident rosacea. J Invest Dermatol. october 2013;133(10):2480-3.
24. Zhao M, Mantel I, Gelize E, Li X, Xie X, Arboleda A, et al. Mineralocorticoid receptor antagonism limits experimental choroidal neovascularization and structural changes associated with neovascular age-related macular degeneration. Nat Commun. 21 2019;10(1):369.
25. Liu S, Richards SM, Lo K, Hatton M, Fay A, Sullivan DA. Changes in gene expression in human meibomian gland dysfunction. Invest Ophthalmol Vis Sci. avr 2011;52(5):2727-40.

1. A method of treating ocular rosacea in a patient in need thereof comprising administering to the patient a therapeutically effective combination comprising at least one mineralocorticoid receptor (MR) antagonist and at least one glucocorticoid receptor (GR) agonist having an enhanced binding affinity, wherein administration of the therapeutically effective combination results in enhanced therapeutic efficacy and reduced side-effects relative to administration of the at least one MR antagonist alone or the at least one GR agonist alone.

2. A method for enhancing the potency of a MR antagonist in a subject suffering from ocular rosacea as part of a treatment regimen, the method comprising administering to the subject a therapeutically effective amount of a GR agonist having an enhanced binding affinity.

3. A method for reducing the side-effects of GR agonists in a subject suffering from ocular rosacea as part of a treatment regimen, the method comprising administering to the subject a therapeutically effective combination comprising at least one MR antagonist and at least one GR agonist having an enhanced binding affinity.

4. The method of claim 1, wherein the at least one MR antagonist is an epoxy-steroidal mineralocorticoid receptor antagonist or a non-epoxy-steroidal mineralocorticoid receptor antagonist.

5. The method of claim 4 wherein the at least one MR antagonist is selected from the group consisting of finerenone, spironolactone, canrenone, potassium canrenoate and eplerenone.

6. The method of claim 1, wherein the at least one MR antagonist is an inhibitor of MR expression.

7. The method of claim 1, wherein the at least one GR agonist having an enhanced binding affinity is selected from the group consisting of: triamcinolone acetonide, RU 24782, RU 24858, RU 40066, RU28362, 11-ketosteroid 11-ketodexamethasone, LGD-5552, ZK 216348, SA22465 and an active metabolite of SA22465.

8. The method of claim 1, wherein the at least one MR antagonist and the at least one GR agonist are administered to the patient separately.

9. The method of claim 1, wherein the at least one MR antagonist and the at least one GR agonist are administered to the patient in the same pharmaceutical composition.

10. The method of claim 1, wherein the at least one MR antagonist and the at least one GR agonist are administered via topical or systemic administration or by loco regional administration.

11. The method of claim 10 wherein the topical administration is carried out by a means selected from passive diffusion, iontophoresis, sonophoresis, electroporation, mechanical pressure, osmotic pressure gradient, occlusive cure, microinjections, by needle-free injections by means of pressure, by microelectric patches, or any combination thereof.

12. The method of claim 1, wherein the at least one MR antagonist and the at least one GR agonist are administered to the patient via a subconjunctival injection.

13. A pharmaceutical composition comprising a combined amount of at least one MR antagonist and at least one GR agonist having an enhanced binding affinity.

14. The pharmaceutical composition according to claim 13 that is a liquid, a paste or a solid.

15. The pharmaceutical composition according to claim 13, wherein the pharmaceutical composition is in the form of a solution, a gel or an emulsion.

16. The method of claim 6, wherein the inhibitor of MR expression is a siRNA or an antisense RNA.

17. The method of claim 7, wherein the active metabolite of SA22465 is SA22313, AL-438, ZK 216348 or BI 115.

18. The method of claim 10, wherein the loco regional administration is sub conjunctival, sub tenon, peri bulbar, latero bulbar, retro bulbar or sub tenon injection or delivery.

19. The pharmaceutical composition according to claim 13, wherein the pharmaceutical composition is in the form of an oil-in-water emulsion, a water-in-oil emulsion, a cream, a lotion, a micro emulsion, a gel, and ointment, liposomes, a powder, an aqueous solution, and aqueous suspension, an aerosol, a spray and a wash.
